(19) United States
(12) Patent Application Publication
(10) Pub. No.: US 2025/0032168 A1
(43) Pub. Date: Jan. 30, 2025

(54) DUAL-MODE TISSUE ABLATION NEEDLE

(71) Applicant: SHENZHEN PULSECARE MEDICAL TECHNOLOGY CO., LTD., Shenzhen, Guangdong (CN)

(72) Inventor: Jianwen TAN, Shenzhen, Guangdong (CN)

(21) Appl. No.: 18/696,643

(22) PCT Filed: Sep. 2, 2022
(86) PCT No.: PCT/CN2022/116737

§ 371 Date: Mar. 28, 2024

(30) Foreign Application Priority Data
Sep. 28, 2021 (CN) 202111144804.5

(51) Int. Cl. A61B 18/14; A61B 18/00

(52) U.S. Cl. CPC A61B 18/1402; A61B 2018/00577; A61B 2018/00714

(57) ABSTRACT

The present application relates to the technical filed of ablation needle, and provides a dual-mode tissue ablation needle including a needle body (1), an insulation assembly (2), and a water delivery assembly (3); the insulation assembly (2) is slidably sleeved outside the needle body (1); a water channel (121) is provided inside the insulation assembly (2), an end of the water channel (121) is in communication with the water delivery assembly (3), and an other end of the water channel (121) is in communication with outside. The present application is simple in structure, which is easy to operate and use, and the structure can be used for dual-mode switching for different lesions and tumors of different sizes, the difficulty and risk of surgery is reduced, and the practicability is strong.

### CROSS REFERENCE TO RELATED APPLICATION

The present application is a National Stage Application of International Patent Application No. PCT/CN2022/116737, filed on Sep. 2, 2022, and claims priority of Chinese Patent Application, with application Ser. No. 20/211,1144804.5, filed on Sep. 28, 2021: the contents each of which are incorporated herein by reference.

### TECHNICAL FIELD

The present application relates to the technical field of ablation needles, and more particularly to a dual-mode tissue ablation needle.

### BACKGROUND

Under the background that minimally invasive treatment methods are gradually accepted by doctors and patients, various tissue ablation techniques are becoming an important means in tumor treatment and other fields. Compared with radio frequency, freezing and microwave ablation, the pulsed electric field ablation is a new ablation method using high-voltage pulsed electric field as energy. The pulsed electric field ablation does not rely on temperature effect, but releases high-voltage electric pulse to form irreversible perforation on the cell membrane and destroy the intracellular balance, thus causing rapid cell apoptosis. At present, the pulsed electric field ablation can mainly be used in the treatment of tumor and atrial fibrillation. When applied to tumor ablation, this technique is also known as “nanoknife” because it can form a nanoscale irreversible perforation on the cell membrane of tumor cells.

When the pulsed electric field ablation is used for tumor ablation, the ablation needle is usually inserted into the lesion to emit high-voltage pulses. The pulsed electric field ablation has the advantages of short ablation time, protecting important tissues such as blood vessels and nerves in the treated area, not being affected by heat pool effect, and clear treatment boundary, etc.

Although the pulsed electric field ablation has the advantages mentioned above, a bipolar pulse is generally required for high-voltage pulse discharge, so two ablation needles need to be inserted at one treatment location. In addition, due to the limitation of the safe peak voltage of the pulse, the spacing of the two ablation needles generally cannot exceed 2 cm. When the tumor to be ablated with a size exceeds 2 cm, more ablation needles need to be inserted, which not only increases the cost of the patient, but also makes it difficult for the doctor to insert needles during the operation, and the surgical risk is increased. Therefore, the pulsed electric field ablation needles are not suitable for ablation of large tumors. In addition, in the process of removing the needles after treatment, the pulsed electric field ablation cannot achieve needle path ablation, so there is a risk of tumor needle path metastasis.

### SUMMARY

Regarding to above situations, in order to overcome the shortcomings of the prior art, the present application provides a dual-mode tissue ablation needle, which can effectively solve the problem that the pulse ablation is not convenient for large tumors and cannot achieve needle path ablation in the previous technique.

An embodiment of the present application provides a dual-mode tissue ablation needle, which includes: a needle body, an insulation assembly slidably, and a water delivery assembly:
the insulation assembly is slidably sleeved outside the needle body:
a water channel is provided inside the insulation assembly, an end of the water channel is in communication with the water delivery assembly, and an other end of the water channel is in communication with outside.

In an embodiment, the needle body includes a needle tip and a needle tube that are integrally connected, the water channel is arranged in the needle tube, and a temperature measuring device is arranged in the water channel.

In an embodiment, the dual-mode tissue ablation needle further includes a handle:
the insulation assembly comprises a sliding mechanism and an insulation sleeve, the sliding mechanism is slidably arranged in the handle, and an end of the needle body penetrates through the handle and the sliding mechanism: and the insulation sleeve is fixedly connected to the sliding mechanism.

In an embodiment, the handle is provided with a sliding groove extending along a radial direction of the needle body, and a sidewall of the sliding groove is provided with a plurality of limiting grooves:
the sliding mechanism comprises: a base, a button, and a clamp pin: and
the base is slidably arranged in the sliding groove, the button is connected with the base through a spring, and the button is able to move on the base along the radial direction of the needle body: and the clamp pin is fixed to the button.

In an embodiment, the sliding mechanism further includes a scale indicator fixed on the base.

In an embodiment, the ablation needle further comprises a needle seat fixedly connected to the handle, the needle body is penetrated through the handle and is in communication with the needle seat, and the water delivery assembly is fixedly connected to the needle seat:
a metal pressure tube is sleeved outside the needle seat, an energy transmission wire is pressed and fixed between the metal pressure tube and an outer wall surface of the needle seat, and the energy transmission wire is configured for receiving a pulse current or a radio frequency energy.

In an embodiment, the water delivery assembly includes a water tube seat, a water delivery tube, and a Ruhr joint:
the water tube seat is sealingly adhered to the needle seat, an end of the water delivery tube is fixed to the water tube seat, and the Ruhr joint is fixed to an other end of the water delivery tube.

In an embodiment, a step hole is arranged in the needle seat, the needle body is fixed to an end of the step hole with a small inner diameter, and the water tube seat is fixed to an end of the step hole with a large inner diameter.

In an embodiment, the insulation assembly further includes an insulation layer covering the temperature measuring device.

In an embodiment, the insulation layer is made of at least one of a polyimide and an epoxy.

In an embodiment, an annular groove is arranged at a joint of the needle tip and the needle tube.

In an embodiment, a sidewall of the needle tube is provided with a water outlet hole in communication with the water channel, and the water outlet hole is located next to the needle tip.

In an embodiment, the dual-mode tissue ablation needle further includes a device host: the device host comprises a temperature measuring master board and a temperature measuring sub-board inserted into the temperature measuring master board; and the temperature measuring device is electrically connected with the temperature measuring sub-board.

In an embodiment, the temperature measuring sub-board includes a temperature measuring front end, a microcontroller unit, an isolated power supply unit, and an isolated communication unit: the temperature measuring front end comprises a pulse protection circuit, a filter circuit, and a digital temperature measuring chip circuit: the microcontroller unit is electrically connected with the digital temperature measuring chip circuit: and the isolated power supply unit is an isolated power supply circuit: and
the temperature measuring master board includes a power supply circuit, an optical fiber transceiver, and a temperature measuring sub-board interface: the power supply circuit is configured for supplying power to the isolated power supply unit: and the optical fiber transceiver communicates with the isolated communication unit through asynchronous transceiver communication.

An embodiment of the present application further provides a method for controlling a temperature of a dual-mode tissue ablation needle applied to the dual-mode tissue ablation needle above mentioned, and the method includes:

setting an allowable value of a needle tip temperature of the dual-mode tissue ablation needle:
setting treatment parameters, wherein the treatment parameters comprise a field strength and a pulse width:
outputting a high-voltage pulse energy to the needle body according to the treatment parameters:
collecting the needle tip temperature of the needle body in a real-time:
determining whether the needle tip temperature exceeds the allowable value of the needle tip temperature:
reducing the pulse width and increasing pulse interval time: and filling a salt water to the water delivery assembly.

The present application addresses the problem that the pulsed ablation in the previous technique is not convenient for large tumors, and has the following beneficial effects:
1. The insulation assembly is arranged outside the needle body, and the water channel is arranged inside the needle body and is in communication with the water delivery assembly, so that the device can apply pulsed electric field and radio frequency energy at the same time, and realize dual-mode tissue ablation of the pulsed electric field and the radio frequency energy, so that the device can still perform surgery conveniently and reduce the difficulty of surgery when facing large-sized tumor tissues:
2. The added radio frequency ablation mode of the device can also ensure that the needle path ablation can be achieved during the needle removing operation, thus preventing the needle path metastasis of the tumor:
3. The insulation sleeve slidably added outside the needle body, so as to realize the adjustable naked length of the needle body, to adapt to different surgical needs:
4. The annular groove is arranged at the needle tip to ensure that the needle body can be stuck tightly after entering the tumor tissue to prevent needle withdrawal caused by muscle contraction during treatment:
5. The temperature measuring assembly is added inside the needle body, so as to realize real-time monitoring of the temperature of the needle body and to effectively prevent the temperature of the needle body from being too high to damage normal tissues, and at the same time, the temperature measuring assembly can also cooperate with the water delivery assembly to achieve closed-loop temperature control:

The present application is simple in structure, which is easy to operate and use, and the structure can be used for dual-mode switching for different lesions and tumors of different sizes, the difficulty and risk of surgery is reduced, and the practicability is strong.

### BRIEF DESCRIPTION OF THE DRAWINGS

In order to explain the embodiments of the present application more clearly, a brief introduction regarding the accompanying drawings that need to be used for describing the embodiments of the present application or the prior art is given below: it is obvious that the accompanying drawings described as follows are only some embodiments of the present application, for those skilled in the art, other drawings can also be obtained according to the current drawings on the premise of paying no creative labor.

FIG. 1 is a front schematic view of a dual-mode tissue ablation needle provided by an embodiment of the present application:

FIG. 2 is a sectional schematic view of a handle part of a dual-mode tissue ablation needle as shown in FIG. 1:

FIG. 3 is a sectional schematic view of Part A of a dual-mode tissue ablation needle as shown in FIG. 1:

FIG. 4 is a sectional schematic view of Part A of a dual-modal tissue ablation needle shown in FIG. 1 in another embodiment:

FIG. 5 is a local sectional schematic view of a sliding assembly in a dual-mode tissue ablation needle as shown in FIG. 1:

FIG. 6 is a sectional schematic view of a needle seat in a dual-mode tissue ablation needle as shown in FIG. 1:

FIG. 7 is a perspective schematic view of a device host of a dual-mode tissue ablation needle device as shown in FIG. 1: and

FIG. 8 is a flowchart of real-time upload of temperature measuring data and temperature control steps in a dual-mode tissue ablation needle provided by an embodiment of the present application.

In the drawings, the reference signs are listed:

1—needle body: 11—needle tip: 12—needle tube: 121—water channel: 122—water outlet hole: 13—needle seat: 131—step hole: 14—annular groove:

2—insulation assembly: 21—insulation sleeve: 22—sliding mechanism: 221—base: 222—button: 223—clamp pin: 224—spring: 225—scale indicator:

3—water delivery assembly: 31—temperature measuring device: 311—insulation layer: 32—water tube seat: 33—water delivery tube: 34—Ruhr joint:

4—handle: 41—sliding groove: 42—limiting groove: 43—rear cover:

5—metal pressure tube; and

6—device host: 61—temperature measuring master board: 611—power supply circuit: 612—optical transceiver: 613—temperature measuring sub-board interface: 62—temperature measuring sub-board: 621—temperature measuring front end: 622—microcontroller unit: 623—isolation power supply unit: 624—isolated communication unit.

### DETAILED DESCRIPTION OF EMBODIMENTS

In order to make the purpose, the technical solution and the advantages of the present application be clearer and more understandable, the present application will be further described in detail below with reference to accompanying figures and embodiments. It should be understood that the specific embodiments described herein are merely intended to illustrate but not to limit the present application.

It is noted that when a component is referred to as being “fixed to” or “disposed on” another component, it can be directly or indirectly on another component. When a component is referred to as being “connected to” another component, it can be directly or indirectly connected to another component. Terms such as “up”, “down”, “left”, “right”, and so on are the directions or location relationships shown in the accompanying figures, which are only intended to describe the present application conveniently and simplify the description, but not to indicate or imply that an indicated device or component must have specific locations or be constructed and manipulated according to specific locations: therefore, these terms shouldn't be considered as any limitation to the present application.

It should also be noted that the same reference sign is used to represent the same component or part in the embodiment of the present application. For the same part in the embodiment of the present application, only one of the parts may be marked in the drawing as an example. It should be understood that the reference sign is also applicable to other identical parts or components.

In order to illustrate the technical scheme described in the present application, the following is illustrated in conjunction with specific drawings and embodiments.

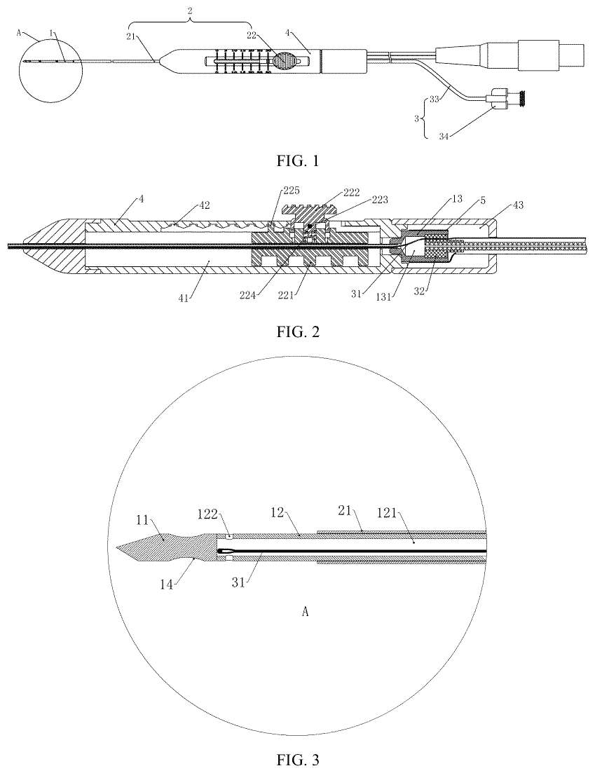

As shown in FIG. 1 to FIG. 3. The embodiment provides a dual-mode tissue ablation needle, which, combined with the host, can realize dual-mode switching of the pulsed electric field and the radio frequency energy on a single needle body 1, so that the device can switch different modes when facing tumors of different sizes, so as to reduce the difficulty and risk of surgery.

The dual-mode tissue ablation needle includes a needle body 1, an insulation assembly 2, and a water delivery assembly 3. One end (the needle tip 11) of the needle body 1 will directly enter the tumor tissue through user operation, and the other end of needle body 1 is connected to an external host, the external host can realize dual-mode switching of the pulsed electric field and the radio frequency energy by adjusting the output energy. The insulation assembly 2 is used to provide insulation protection for the needle body I when the device is in the pulse mode, and the water delivery assembly 3 is used to provide cooling for the needle body 1 when the device is in the radio frequency mode.

The insulation assembly 2 is slidably sleeved outside the needle body 1, and the insulation assembly 2 can slide along the axial direction of the needle body 1 and surround the sidewall surface of the needle body 1 in a 360°. The insulation assembly 2 cannot completely surround the entire sidewall of the needle body I at the same time, that is, the needle body 1 and the tumor tissue cannot be directly contacted where the insulation assembly 2 is located. Moreover, the electric field formed by the high-voltage pulse cannot act on the tumor tissue where the insulation assembly 2 is located. When the device is in the pulse mode, the naked length of needle body I can be adjusted by sliding the position of the insulation assembly 2, which can adapt to various tumor tissues of different sizes and prevent other normal tissues outside the tumor tissues from contacting the needle body 1 and causing damage.

As shown in FIG. 3, a water channel 121 is provided inside the needle body 1, and one end of the water channel 121 is in communication with the water delivery assembly 3, and the end is the inlet end. The water delivery assembly 3 is in communication with the external water source, and the external water source is mainly used to supply water to the water delivery assembly 3. The external water source can include a power pump, a water tank, or other common water sources. The power pump can pump the liquid in the water tank into the water channel 121 through the water delivery assembly. The liquid contained in the water tank can be normal saline or muscle relaxant: the other end of the needle body 1 is in communication with the outside, and the other end of the needle body 1 is provided with a water outlet hole 122. The liquid in the water channel 121 flows out through the water outlet hole 122. The length of the water channel 121 is similar to the length of the needle body 1, so as to ensure that the cooling liquid can reduce the overall temperature of the needle body 1 after entering the water channel 121.

When the device is in the radio frequency mode, the water delivery assembly 3 is used to inject the liquid into the entire needle body 1 through the water channel 121, which is used to reduce the temperature near the tumor tissue and the needle body 1, and prevent the burn caused by excessive temperature at the needle body 1.

In the embodiment, when the ablation needle is in the pulse mode, part of the needle body 1 is protected by the insulation assembly 2 to prevent the part of the needle body 1 outside the tumor tissue from destroying other tissues outside the tumor tissue. When the ablation needle is in the radio frequency mode, the water channel 121 is in communication with the external water source through the water delivery module 3, so as to cool down the tumor tissue near the needle body 1 and the needle body 1. That is, in the embodiment, the structural support for the pulse mode is achieved by providing the insulation assembly 2, and the structural support for the radio frequency mode is achieved by providing the water delivery assembly 3 and water channel 121, so that the ablation needle can adapt to dual-mode tissue ablation of the pulsed electric field and the radio frequency energy. In the face of large tumor tissue size, the ablation needle provided in the embodiment can easily switch modes and achieve surgery, the difficulty of surgery is reduced.

Further, the needle body 1 is made of material with good biocompatibility, high hardness, high sharpness and high heat resistance. The material with good biocompatibility can reduce the uncontrollable changes of the tumor when the needle body I is inserted into the tumor, and the high hardness and high sharpness can ensure that needle body I enters the tumor tissue quickly and accurately. The high heat resistance is to prevent the deformation or other uncontrollable changes of the needle body I caused by the high temperature for a long time in the radio frequency state.

Further, the material of the needle body 1 is a medical stainless steel, such material can effectively meet the requirements of the needle body I for biocompatibility, hardness, sharpness and heat resistance. At the same time, the material itself has a good rust prevention ability, which can prevent the rust that may occur in the long-term wet environment of the needle body 1.

Further, hydrophobic anti-rust treatment is applied to the outer wall surface of the needle body 1 and the inner wall surface of the water channel 121. The hydrophobic treatment on the outer wall surface of the needle body I can make it easier for the needle body I to penetrate further after entering the tumor tissue, the friction between the tumor tissue and the wall surface of the needle body 1 is reduced, and the possibility of stagnation is reduced. The hydrophobic treatment of the inner wall surface of the water channel 121 is mainly used to prevent the long-term accumulation and adhesion of the cooling liquid in the sidewall of the water channel 121, and the hydrophobic treatment inside and outside the needle body 1 is also convenient for the cleaning of the needle body 1.

Further, the outer wall surface of the needle body 1 is coated with an insulation coating, and the insulation coating can be made of at least one of polyimide, teflon or perelin. and the thickness of the coating is more than 40 μm, for example, the thickness can be 40 μm, 45 μm, 50 μm or other thickness dimensions to achieve a voltage resistance of more than 3 KV.

Further, in the embodiment, there are two technical solutions for the cooling of the water delivery assembly 3 that can both be used and achieve the cooling effect, as follows:

The first type: the water inlet end is in communication with the water delivery assembly 3, the water outlet hole 122 is in communication with the outside, and when the needle body I enters the tumor tissue, the water outlet hole 122 is not in the tumor tissue, that is, the water outlet hole 122 is relatively close to the water inlet end: at this time, when the water channel 121 is filled with liquid, the liquid will flow directly from the water outlet hole 122 to the outside, or the liquid will be cooled and collected and re-filled into the water tank. In this way, the internal circulation of liquid can be realized.

The second type: the water inlet end is in communication with the water delivery assembly 3, the water outlet hole 122 is in communication with the outside, and when the needle body I enters the tumor tissue, the water outlet hole 122 then enters the tumor tissue, that is, the water outlet hole 122 is relatively close to the needle tip 11 of the needle body 1. The liquid flows through the whole water channel 121 and exits from the water outlet hole 122 after the liquid enters the water channel 121 through the water inlet end: at this time, the outflow liquid enters the tumor tissue to achieve the effect of cooling the tissue near the needle body I and the needle body 1. In this method, the water delivery assembly 3 provides a small water pressure, and the outflow liquid from the water outlet hole 122 only achieves the cooling effect near the needle body 1.

It should be noted that in the present embodiment, when the needle body I enters the tumor tissue in pulse mode, a small amount of tissue fluid will flow into the water channel 121 through the water outlet hole 122, but limited by the internal pressure of the tumor tissue, the tissue fluid entering the water channel 121 is not much and the tissue fluid will not flow upstream along the water channel 121, and the subsequent use of the device will not be affected.

For specific use in the embodiment, the needle body I is pushed into the designated position of the tumor tissue, and then the position of the insulation assembly 2 is adjusted first if the pulse mode is required, and then the needle body 1 releases the high-voltage pulse by the host. If the radio frequency mode is required, the water delivery assembly 3 is activated first, and then the needle body 1 is heated by the host. Thus, it can be ensured that there is a cooling liquid in the water channel 121 and a certain cooling liquid outside the needle body 1 when the needle body 1 is heating up.

It should be noted that when the needle is retreated and the pulse mode ends, it is necessary to switch to the radio frequency mode during the process of retreating the needle to ensure the ablation of the needle path after retreating the needle and prevent the needle path metastasis of the tumor.

As shown in FIG. 2 and FIG. 3, the needle body I includes a needle tip 11 and a needle tube 12 connected together. The needle tip 11 is convenient for the needle body 1 to enter tumor tissue, and one end of the needle body I is connected with the water delivery assembly 3, the water channel 121 is arranged in the needle tube 12, the temperature measuring device 31 is arranged inside the water channel 121, and the temperature measuring device 31 is used for monitoring the temperature of the cooling liquid in the watercourse 121 in real time: the temperature measuring device 31 can be a common temperature sensor, a temperature measuring wire or other devices that can achieve the temperature measuring effect. The temperature measuring device 31 is electrically connected with the external control device. The user can determine the cooling condition of the needle body 1 through the real-time temperature monitored by the temperature measuring device 31, so as to facilitate the user to control the cooling effect through the water flow or flow speed, and realize the closed-loop control of the temperature of the needle body 1.

In one embodiment, the temperature measuring device 31 is coated with an insulation layer 311, the insulation layer 311 is made of at least one of polyimide and epoxy. The insulation layer 311 is arranged to ensure the insulation between the temperature measuring wire and the needle body 1, and the insulation layer 311 has good thermal conductivity. When the ablation needle is in the pulse mode, the needle body I releases high-voltage pulse energy for tissue ablation, the high-voltage pulse energy will cause damage to the temperature measuring device 31. The insulation layer 311 can effectively protect the temperature measuring device 31.

As shown in FIG. 3, further, the temperature measuring device 31 is a temperature measuring wire, and the insulation layer 311 covers the temperature measuring wire. One end of the temperature measuring wire is fixed at the end of the water channel 121, and the other end extends out of the water channel 121. The temperature measuring wire is more easily adapted to the narrow environment inside the water channel 121, and can monitor the temperature without obstructing the normal flow of the liquid. Specifically, the sidewall of the water delivery tube 33 is provided with a small hole, and the temperature measuring wire is extended to the outside through the small hole, and the small hole is sealed by a sealant, so as to separate the temperature measuring wire from the liquid in the water delivery tube 33.

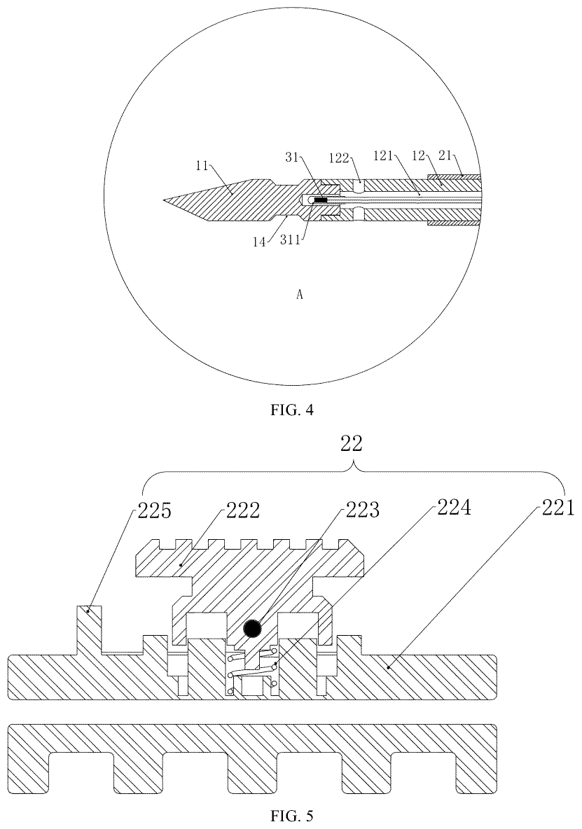

As shown in FIG. 4, further, the temperature measuring device 31 is a temperature sensor, one end of the temperature sensor is embedded in the needle tip 11 towards one end of the water channel 121, the other end of the temperature sensor is placed in the water channel 121, and the temperature sensor is covered with the insulation layer 311 to protect the temperature sensor.

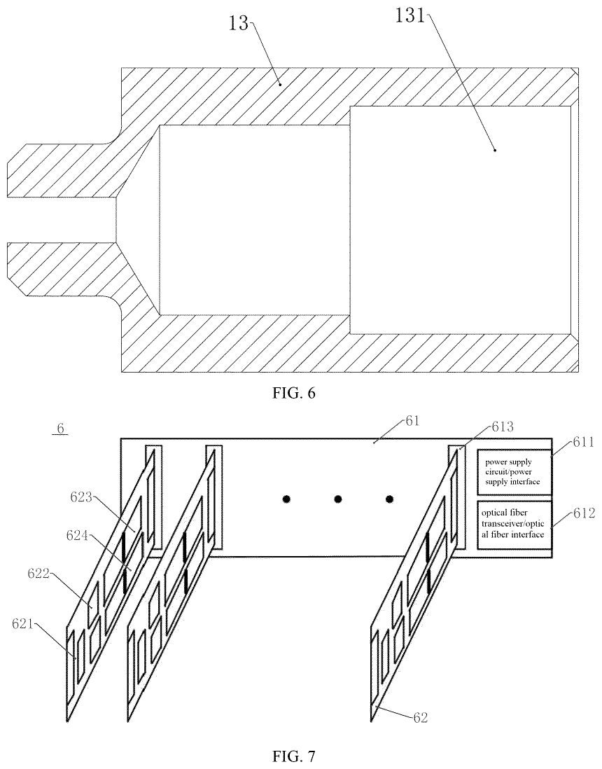

As shown in FIG. 3 to FIG. 5, the ablation needle in the embodiment of the present application further includes a handle 4, one end of the needle body I away from the tip 11 fixed in the handle 4, and the handle 4 is arranged to facilitate the user to hold the device and provide a fixed basis for the subsequent structure.

The insulation assembly 2 includes a sliding mechanism 22 and an insulation sleeve 21, the sliding mechanism 22 is placed in the handle 4, the insulation sleeve 21 is slidably sleeved outside the needle tube 12, one end of the insulation sleeve 21 is fixedly connected with the sliding mechanism 22, and the sliding mechanism 22 can drive the needle tube 12 to slide synchronously. The sliding mechanism 22 can be any common structure that can slide along the handle 4, such as a sliding block, a sliding sleeve, etc. The function of the sliding mechanism 22 is only to drive the insulation sleeve 21 to move in an axial direction along the needle tube 12, and the length of the insulation sleeve 21 is less than the length of the needle tube 12, so as to leave room for the sliding mechanism 22 to adjust the position of the insulation sleeve 21. One end of the needle body I passes through the handle 4, the insulation sleeve 21 and the sliding mechanism 22, and one end of the needle tube 12 passes through the handle 4, the insulation sleeve 21 and the sliding mechanism 22 and is fixed at the end of the handle 4, and is in communication with the water delivery assembly 3, so that the sliding of the insulation sleeve 21 will not drive the needle tube 12 to move.

As shown in FIG. 2 to FIG. 5, further, a sliding groove 41 is provided on the handle 4, and the sliding groove 41 extends along the axial direction of the needle body 1: the top surface of the sliding groove 41 is provided with an opening to enable the sliding groove 41 to in communication with the outside, and the sidewall of the sliding groove 41 is provided with a plurality of limiting grooves 42.

The sliding mechanism 22 includes a base 221, a button 222 and a clamp pin 223. The base 221 is slidably arranged in the sliding groove 41, the base 221 can slide along the axial direction of the needle tube 12 in the sliding groove 41, the button 222 and the base 221 are connected by a spring 224, and the button 222 is slidably connected on the base 221 along a radial direction of the needle tube 12. The upper end of the button 222 is placed outside the handle 4 through the opening to facilitate the user to push and press, the clamp pin 223 is fixed on the button 222, the up and down movement of the button 222 can drive the clamp pin 223 to synchronously move, and the clamp pin 223 can be placed in the limiting groove 42.

Further, the above technical solution further includes the following technical solutions of sliding mechanism 22, in particular:

The first type: in the resetting process of the spring 224, the button 222 is driven to move in a direction away from the needle tube 12; in the technical solution, the button 222 is pressed to drive the clamp pin 223 out from the limiting groove 42:

The second type: in the resetting process of the spring 224, the button 222 is driven to move in a direction away from the needle tube 12: in the technical solution, the button 222 is raised to drive the clamp pin 223 out from the limiting groove 42:

The third type: the button 222 is connected to the clamp pin 223 through the spring 224, and button 222 does not move radially along the needle tube 12; in the technical solution, the button 222 is pushed to move along the axial direction of the needle tube 12, and the clamp pin 223 is ejected from the limiting groove 42. When the button 222 is stop pushing. the clamp pin 223 is stuck into the corresponding limiting groove 42 under the action of the spring 224.

The above technical solutions are used to realize the position adjustment and position fixation of the insulation sleeve 21.

As shown in FIG. 2, further, a guide cone is arranged between adjacent limiting grooves 42. The guide cone consists of two intersecting incline surfaces and the intersection is the tip of the guide cone. The direction of the guide cone from tip to seat is the resetting direction of the spring 224. The guide cone is provided to assist the clamp pin 223 to enter the limiting groove 42: and when the user does not push the button 222 exactly to the limiting groove 42, during the resetting process of the spring 224, the clamp pin 223 first contacts the inclined surface of the guide cone, and finally enters the limiting groove 42 under the guiding action of the inclined surface.

Further, the outer wall surface of the handle 4 is further provided with scale lines and identification number corresponding to each limiting groove 42, and a scale indicator is fixed on the base 221. When the clamp pin 223 falls into a limiting groove 42, the scale indicator 225 precisely points to the corresponding scale.

In the embodiment, when the device is in the radio frequency mode and the user needs to adjust the position of the insulation sleeve 21, the user can press the button 222 and push the button 222 to push the base 221 to slide in the sliding groove 41, while paying attention to the scale indicator 225 and the scale, and releasing the button 222 when the specified scale is reached.

As shown in FIG. 2, in Embodiment 1, the ablation needle further includes a needle seat 13 fixed on the handle 4, and the needle seat 13 is fixed on one end of the handle 4 away from the needle tip 11, the needle body 1 penetrates through the handle 4 and connects to the needle seat 13, the water delivery assembly 3 is fixed on the needle seat 13, a through hole is provided inside the needle seat 13, and needle seat 13 is used to fix the needle body 1, and the needle body I is in communication with the through hole in the needle seat 13.

The water delivery assembly 3 includes a water tube seat 32, a water delivery tube 33, and a Ruhr joint 34: the water tube seat 32 is sealingly adhered to the needle seat 13: the water channel 121, the through hole and water tube seat 32 are in communication with each other herein, one end of water delivery tube 33 is fixed to the water tube seat 32, the Ruhr joint 34 is fixed to the other end of the water delivery tube 33, and the Ruhr joint 34 is connected to an external water source. The Ruhr joint 34 can accommodate common medical and laboratory equipment, and the external water source enters the water channel 121 through the Ruhr joint 34, the water tube seat 32, and the needle seat 13. The external water source can be a combination of a water supply pump and a water tank, or other water source with water supply capacity.

A step hole 131 is provided in the needle seat 13, the step hole 131 is a through hole, and the diameters of the opposite ends of the step hole 131 are different from each other. The end of step hole 131 with a smallest diameter is in communication with the needle tube 12, and the end of step hole 131 with a largest diameter is in communication with the water tube seat 32. The outer wall surface of the end with a larger diameter of the step hole 131 is provided with knurl and is sleeved with a metal pressure tube 5. The metal pressure tube 5 presses the energy transmission wire onto the outer wall surface of the end with the larger diameter of the needle seat 13. The material of the metal pressure tube 5 can be brass, stainless steel, nickel-plated copper, or other common metal materials with good electrical conductivity and certain elastic deformation ability. The elastic deformation ability can ensure that the metal pressure tube 5 can more firmly press the energy transmission wire onto the needle seat 13.

Further, one end of the handle 4 is fastened to a rear cover 43 located outside the needle seat 13, and the energy transmission wire and the cable of the temperature measuring device 31 are extended to the outside through the rear cover 43 and integrated into a special connector connected to the host to provide pulse or radio frequency energy to the needle body 1.

As shown in FIG. 3 and FIG. 4, in Embodiment 1, an annular groove 14 is arranged at the joint of the needle tip 11 and the needle tube 12. The annular groove 14 is arranged to prevent the needle from retreating caused by muscle contraction during treatment, that is, when the muscle contracts, part of the muscle tissue will enter the annular groove 14, so as to clamp the needle body 1 and to prevent the needle body 1 from being squeezed out.

As shown in FIG. 7, the ablation needle in the embodiment of the present application further includes a device host 6, the device host 6 is used to control the radio frequency mode or pulse mode of the ablation needle and provide radio frequency energy or pulse energy for the ablation needle, and the device host 6 is further used to control the temperature of the ablation needle.

The device host 6 includes a temperature measuring master board 61 and a temperature measuring sub-board 62, and the temperature measuring sub-board 62 is electrically connected with the temperature measuring master board 61: the temperature measuring master board 61 includes a power supply circuit 611, a fiber optic transceiver 612, and a temperature measuring sub-board interface 613: the temperature measuring sub-board 62 includes a temperature measuring front end 621, a microcontroller unit 622, an isolated power supply unit 623, and an isolated communication unit 624.

The power supply circuit 611 is used to supply power to the isolated power supply unit 623: the optical fiber transceiver 612 is used for data transmission. The optical fiber transceiver 612 can receive the data sent by the isolated communication unit 624 and send the data to the control unit of the device host 6; and the temperature measuring sub-board interface 613 is used to connect the temperature measuring sub-board 62 to the temperature measuring master board 61.

The temperature measuring front end 621 is electrically connected with the temperature measuring device 31. The temperature measuring front end 621 includes a pulse protection circuit, a filter circuit, and a digital temperature measuring chip circuit: and the pulse protection circuit is used to prevent the high frequency and high voltage pulse energy on the needle body I under the pulse mode from damaging the electronic circuit and electronic components on the temperature measuring master board 61 and temperature measuring sub-board 62. The filter circuit is used to reduce the AC component of the pulse current in the pulse mode, and retain the DC component to reduce the ripple coefficient of the output voltage and make the waveform smoother. The digital temperature measuring chip circuit is used to receive the real-time monitoring signal of the temperature measuring device 31 and convert the signal into data to send to the microcontroller unit 622. The microcontroller unit 622 is used to communicate with the digital temperature measuring chip circuit and set the compensation value of the cold end. The address of the microcontroller unit 622 is set by dip switch. The isolation power supply unit 623 is the isolation power supply circuit, which is specifically the isolation power supply circuit of magnetic isolation. The isolated communication unit 624 is connected to the optical fiber transceiver 612, in particular, the isolated communication unit 624 realizes the universal asynchronous transceiver communication through an optocoupler.

The beneficial effects of the embodiment are:

1. The device host 6 adopts the design of a master board and a sub-board including a temperature measuring master board 61 and a temperature measuring sub-board 62, so that a plurality of temperature measuring sub-boards 62 are electrically connected to the temperature measuring master board 61, and a plurality of temperature measuring sub-circuits are respectively arranged on the plurality of temperature measuring sub-boards 62, the plurality of temperature measuring sub-circuits are used to monitor the temperature of the ablation electrode needle and output temperature data. The temperature measuring master board 61 receives temperature data and sends it to the main control unit to monitor the temperature of ablation needles in real time. The design of the master board and the sub-board can monitor the temperature of a plurality of ablation needles in real time, which can monitor tissue temperature more sensitive and improve the treatment safety of tissue ablation. Moreover, the design of the master board and the sub-board is convenient to control and install the number of the plurality of temperature measuring sub-board 62:

2. The isolation power supply unit 623 is arranged, and the isolation power supply unit 623 adopts the isolation mode to realize the power supply connection between the temperature measuring master board 61 and the temperature measuring sub-circuit, so as to prevent the high voltage conduction between the temperature measuring sub-circuit and the temperature measuring master board 61 due to the power supply connection from damaging the circuit:

3. The isolation communication unit 624 is arranged, and the isolation communication unit 624 is connected with microcontroller unit 622 and the temperature measuring master board 61. The isolated communication unit 624 is used for isolated communication between the temperature measuring sub-circuit and the temperature measuring master board 61, that is, the communication data is transmitted between the temperature measuring sub-circuit and the temperature measuring master board 61 through the isolated communication unit 624. The transmission of the communication data includes the transmission of temperature data, the transmission of cold end compensation value setting information, etc., and the transmission adopts isolated communication mode to prevent high voltage conduction between the temperature measuring sub-circuit and the temperature measuring master board 61 due to the communication connection from damaging the circuit.

In one embodiment, the device host 6 also includes a control unit for controlling parameters related to the radio frequency mode or the pulse mode of the ablation needle.

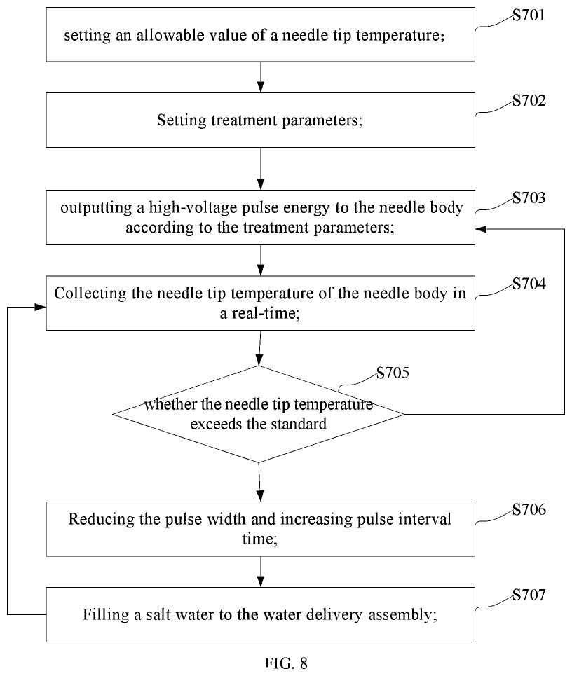

As shown in FIG. 8, the present embodiment further provides a method for controlling a temperature of a dual-mode tissue ablation needle, the method is used to measure and control tissue temperature rise when the ablation needle is in pulse mode, and the method includes the following steps:

In step S701, setting an allowable value of a needle tip temperature of the dual-mode tissue ablation needle.

This step is used before the start of treatment. Before the start of treatment, the allowable value of the needle tip temperature of the dual-mode tissue ablation needle is set. and the allowable value of the needle tip temperature is less than or equal to the allowable value of the tissue temperature.

In step S702, setting treatment parameters.

This step is used to set the treatment parameters, such as field strength, pulse width, etc., before the start of treatment.

In step S703, outputting a high-voltage pulse energy to the needle body 1 according to the treatment parameters.

This step is used for the control unit to output high-voltage pulse energy and perform pulse ablation after the needle body 1 enters the target position in the patient.

In step S704, collecting the needle tip temperature of the needle body in a real-time.

In this step, the temperature measuring device 31 collects the real-time temperature near the needle tip 11 in real time, and specifically uploates the real-time temperature to the device host 6 through the isolated communication unit 624 and the optical fiber transceiver 612.

In step S705, determining whether the needle tip temperature exceeds the standard, that is, determining whether the needle tip temperature exceeds the allowable value of the needle tip temperature.

Then the real-time temperature is compared with the allowable value. If the real-time temperature is greater than the allowable value, then entering the step S705 if the real-time temperature is less than or equal to the allowable value, then returning to the step S703.

In step S706, reducing the pulse width and increasing pulse interval time.

This step is used when the real-time temperature in the step S705 exceeds the allowable value of the needle tip temperature, then the control unit adjusts the pulse interval time and the pulse width of the output pulse, specifically increasing the pulse interval time and decreasing the pulse width to reduce the temperature rise. Optionally, when the real-time temperature change rate is close to 10% of the allowable value, the control unit will also adjust the pulse interval time and the pulse width of the output pulse, specifically increasing the pulse interval time and decreasing the pulse width to reduce the temperature rise.

It should be noted that if the real-time temperature is greater than 20% of the allowable value, the control unit will stop the pulse output to ensure the safety of treatment.

In step S707, filling a salt water to the water delivery assembly 3.

This step is used to increase the flow rate of the salt water in the water channel 121 and to improve the cooling effect by adjusting the water pressure of the external water source.

The beneficial effect of the embodiment is to provide a closed-loop control solution of the ablation needle to ensure that the ablation needle will not burn the tissue of the patient when the ablation needle is in the pulse mode, thus ensuring the safety of the treatment process.

The above embodiments are only intended to explain but not to limit the technical solutions of the present application. Although the present application has been explained in detail with reference to the above-described embodiments, it should be understood for the ordinary skilled one in the art that, the technical solutions described in each of the above-described embodiments can still be amended, or some technical features in the technical solutions can be replaced equivalently; these amendments or equivalent replacements, which won't make the essence of corresponding technical solution to be broken away from the spirit and the scope of the technical solution in various embodiments of the present application, should all be included in the protection scope of the present application.

1. A dual-mode tissue ablation needle, configured for realizing dual-mode tissue ablation of a pulsed electric field and a radio frequency energy, comprising:
a needle body:
an insulation assembly, slidably sleeved outside the needle body: and
a water delivery assembly:
wherein a water channel is provided inside the insulation assembly, an end of the water channel is in communication with the water delivery assembly, and an other end of the water channel is in communication with an outside.

2. The dual-mode tissue ablation needle according to claim 1, wherein the needle body comprises a needle tip and a needle tube that are integrally connected, the water channel is arranged in the needle tube, and a temperature measuring device is arranged in the water channel, wherein the temperature measuring device is an insulation and high pressure resistant temperature measuring device.

3. The dual-mode tissue ablation needle according to claim 2, further comprising a handle:
wherein the insulation assembly comprises a sliding mechanism and an insulation sleeve, the sliding mechanism is slidably arranged in the handle, and an end of the needle body penetrates through the handle and the sliding mechanism; and
the insulation sleeve is fixedly connected to the sliding mechanism.

4. The dual-mode tissue ablation needle according to claim 3, wherein the handle is provided with a sliding groove extending along a radial direction of the needle body, and a sidewall of the sliding groove is provided with a plurality of limiting grooves:
the sliding mechanism comprises: a base, a button, and a clamp pin: and
the base is slidably arranged in the sliding groove, the button is connected with the base through a spring, and the button is able to move on the base along the radial direction of the needle body: and the clamp pin is fixed to the button.

5. The dual-mode tissue ablation needle according to claim 4, wherein the sliding mechanism further comprises a scale indicator fixed on the base.

6. The dual-mode tissue ablation needle according to claim 3, wherein the ablation needle further comprises a needle seat fixedly connected to the handle, the needle body is penetrated through the handle and is in communication with the needle seat, and the water delivery assembly is fixedly connected to the needle seat; and
a metal pressure tube is sleeved outside the needle seat, an energy transmission wire is pressed and fixed between the metal pressure tube and an outer wall surface of the needle seat, and the energy transmission wire is configured for receiving a pulse current or a radio frequency energy.

7. The dual-mode tissue ablation needle according to claim 6, wherein the water delivery assembly comprises a water tube seat, a water delivery tube and a Ruhr joint:
the water tube seat is sealingly adhered to the needle seat, an end of the water delivery tube is fixed to the water tube seat, and the Ruhr joint is fixed to an other end of the water delivery tube.

8. The dual-mode tissue ablation needle according to claim 7, wherein a step hole is arranged in the needle seat, the needle body is fixed to an end of the step hole with a small inner diameter, and the water tube seat is fixed to an end of the step hole with a large inner diameter.

9. The dual-mode tissue ablation needle according to claim 7, wherein the insulation assembly further comprises an insulation layer covering the temperature measuring device.

10. The dual-mode tissue ablation needle according to claim 9, wherein the insulation layer is made of at least one of a polyimide and an epoxy.

11. The dual-mode tissue ablation needle according to claim 2, wherein an annular groove is arranged at a joint of the needle tip and the needle tube.

12. The dual-mode tissue ablation needle according to claim 11, wherein a sidewall of the needle tube is provided with a water outlet hole in communication with the water channel, and the water outlet hole is located next to the needle tip.

13. The dual-mode tissue ablation needle according to claim 1, wherein the dual-mode tissue ablation needle further comprises a device host: the device host comprises a temperature measuring master board and a temperature measuring sub-board inserted into the temperature measuring master board: and the temperature measuring master board is electrically connected with the temperature measuring sub-board.

14. The dual-mode tissue ablation needle according to claim 13, wherein the temperature measuring sub-board comprises a temperature measuring front end, a microcontroller unit, an isolated power supply unit, and an isolated communication unit:
the temperature measuring front end comprises a pulse protection circuit, a filter circuit, and a digital temperature measuring chip circuit: the microcontroller unit is electrically connected with the digital temperature measuring chip circuit: and the isolated power supply unit is an isolated power supply circuit: and the temperature measuring master board comprises a power supply circuit, an optical fiber transceiver, and a temperature measuring sub-board interface: the power supply circuit is configured for supplying power to the isolated power supply unit: and the optical fiber transceiver communicates with the isolated communication unit through an asynchronous transceiver communication.

15. A method for controlling a temperature of a dual-mode tissue ablation needle, applied to a dual-mode tissue ablation needle comprising:
a needle body;
an insulation assembly, slidably sleeved outside the needle body; and
a water delivery assembly;
wherein a water channel is provided inside the insulation assembly, an end of the water channel is in communication with the water delivery assembly, and an other end of the water channel is in communication with an outside; and
wherein the method comprisinges:
setting an allowable value of a needle tip temperature of the dual-mode tissue ablation needle:
setting treatment parameters, wherein the treatment parameters comprise a field strength and a pulse width:
outputting a high-voltage pulse energy to the needle body according to the treatment parameters:
collecting the needle tip temperature of the needle body in a real-time:
determining whether the needle tip temperature exceeds the allowable value of the needle tip temperature:
reducing the pulse width and increasing pulse interval time: and
filling a salt water to the water delivery assembly.

16. The dual-mode tissue ablation needle according to claim 11, wherein a sidewall of the needle tube is provided with a water outlet hole in communication with the water channel, and the water outlet hole is located next to the needle tip.

17. The method according to claim 15, wherein the needle body comprises a needle tip and a needle tube that are integrally connected, the water channel is arranged in the needle tube, and a temperature measuring device is arranged in the water channel.

18. The method according to claim 17, wherein the dual-mode tissue ablation needle further comprises a handle;
wherein the insulation assembly comprises a sliding mechanism and an insulation sleeve, the sliding mechanism is slidably arranged in the handle, and an end of the needle body penetrates through the handle and the sliding mechanism; and
the insulation sleeve is fixedly connected to the sliding mechanism.

19. The method according to claim 18, wherein the handle is provided with a sliding groove extending along a radial direction of the needle body, and a sidewall of the sliding groove is provided with a plurality of limiting grooves;
the sliding mechanism comprises: a base, a button, and a clamp pin; and
the base is slidably arranged in the sliding groove, the button is connected with the base through a spring, and the button is able to move on the base along the radial direction of the needle body; and the clamp pin is fixed to the button.

20. The method according to claim 19, wherein the sliding mechanism further comprises a scale indicator fixed on the base.
